(19) United States
(12) Patent Application Publication
(10) Pub. No.: US 2014/0287070 A1
(43) Pub. Date: Sep. 25, 2014

(54) Dirty Dog Anti-Bacterial Paw Spritz

(71) Applicant: Bonnie Ann Bruderer, Sausalito, CA (US)

(72) Inventor: Bonnie Ann Bruderer, Sausalito, CA (US)

(21) Appl. No.: 13/674,991

(22) Filed: Nov. 13, 2012

(51) Int. Cl. A61K 36/906; A61K 36/889; A61K 36/736; A61K 36/185; A01K 13/00; A61K 36/752

(52) U.S. Cl. CPC A61K 36/906; A01K 13/00; A61K 36/752; A61K 36/736; A61K 36/185; A61K 36/889
USPC 424/727

(57) ABSTRACT

Dirty Dog Anti-Bacterial Paw Spritz is an all-natural way to cut down on germs and bacteria found on dogs paws. It is sprayed on the dog's paws after being exposed to dirt, germs and bacteria. The main key components in Dirty Dog Anti-Bacterial Paw Spritz are ginger and lemon essential oils, which have been proven to cut down on dirt and germs. This heals to protect the dog owner, their home and their family from many illness that are otherwise spread from these germs.

### SUMMARY OF THE INVENTION

Patent Application for Dirty Dog Anti-Bacterial Spray-Reference #30766906

Dirty Dog Anti-Bacterial Paw Spray is an all-natural spray for dogs paws that will cut down on dirt, germs and bacteria, that could make the owner sick.

### BRIEF DESCRIPTION OF THE DRAWINGS

FIG. 1 illustrates a 2.0 oz spray bottle of Dirty Dog Anti-bacterial Paw Spritz.

### DETAILED DESCRIPTION OF THE INVENTION

Dirty Dog Anti-Bacterial Paw Spray is an all-natural spray for paws of a Dog that will cut down on dirt, germs and bacteria, that could make the owner sick.

It is made from water, ginger essential oil, lemon essential oil, cherry essential oil, sesame oil and coconut oil.

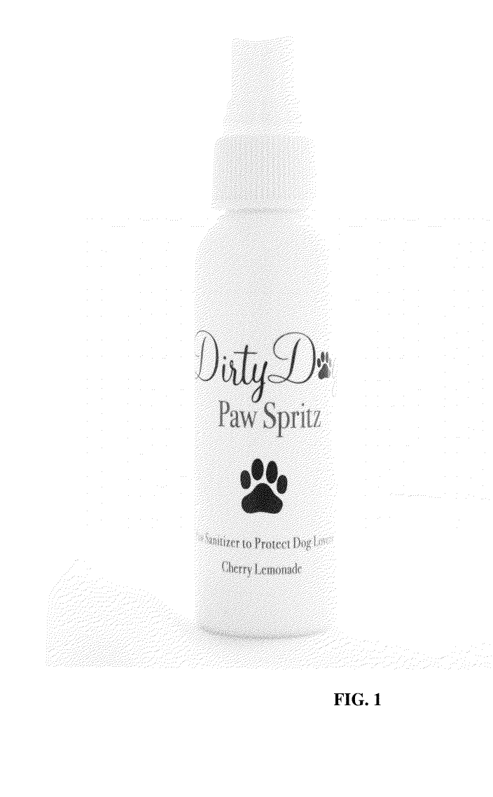

It is sprayed on the paws of a Dog after it has been out running around and exposed to germs. FIG. 1 illustrates a 2.0 oz spray bottle containing Dirty Dog Anti-bacterial Paw Spritz which can be used to spray on the paws of the dog.

I claim:

1. An anti-bacterial composition comprising:
ginger essential oil; and
lemon essential oil.

2. The anti-bacterial composition of claim 1, wherein the anti-bacterial composition is anti-microbial.

3. A method of use of an anti-bacterial composition, comprising:
providing said anti-bacterial composition in a container with a spray means, said antimicrobial composition comprising:
ginger essential oil; and
lemon essential oil;
spraying an effective amount of said anti-bacterial composition on one or more paws of a dog.

4. The method of use of an anti-bacterial composition of claim 3, wherein said anti-bacterial composition reduces amount of dirt, germs and bacteria accumulation in said one or more paws sprayed with said effective amount of said anti-bacterial composition.

5. An anti-bacterial composition comprising:
ginger essential oil;
lemon essential oil;
cherry essential oil,
sesame oil;
coconut oil; and
water.
